(19) United States
(12) Patent Application Publication
(10) Pub. No.: US 2009/0088765 A1
(43) Pub. Date: Apr. 2, 2009

(54) Spinal Interbody Distractor

(76) Inventors: Michael S. Butler, Charles, IL (US); Brian D. Hartsell, Aurora, IL (US)

Correspondence Address: Bruce J. Bowman;Bowman & Davis, P.A., Suite 106, 1016 3rd Ave. SW, Carmel, IN, 46032, US

(21) Appl. No.: 12/239,767

(22) Filed: Sep. 27, 2008

Related U.S. Application Data
(60) Provisional application No. 60/995,538, filed on Sep. 27, 2007.

(51) Int. Cl. A61B 17/58; A61F 2/44

(52) U.S. Cl. 606/90; 623/17.11

(57) ABSTRACT

A spinal interbody distractor is provided that is configured to provide distraction of adjacent vertebrae after implantation within a spinal disc cavity through manual rotation of the distractor once implanted. Such implantation is preferably, but not necessarily, through minimally invasive surgical techniques. In one form, the distractor is configured to be rotated 90° to 180° after implantation to provide distraction of adjacent vertebrae. The distractor has a lateral side to lateral side height of a first length and a superior end to inferior end height of a second length that is greater than the first length. This configuration allows the distractor to be inserted into a spinal disc cavity having an opening with a height that is smaller than an end height of the spinal disc cavity after implantation thereof, thereby providing distraction of adjacent vertebrae. The spinal interbody distractor may have one or more cavities, areas, openings and/or bores for spinal fusion material when the distractor is used as a fusion body. The spinal interbody distractor is made from a biocompatible material such as a thermoplastic (e.g. PEEK), a polymer, metal, combination thereof or otherwise, such as desired and/or is appropriate.

### RELATED APPLICATIONS

This patent application claims the benefit of and/or priority to U.S. Provisional Patent Application Ser. No. 60/995,538 filed Sep. 27, 2007, entitled “Spinal Interbody Distractor” the entire contents of which is specifically incorporated herein by this reference.

### BACKGROUND OF THE INVENTION

1. Field of the Invention

The present invention relates to spinal interbody devices for implantation between a pair of adjacent vertebrae in order to provide distraction and support to the adjacent vertebrae and/or promote bone fusion between the adjacent vertebrae.

2. Background Information

The disc between vertebrae of a human spine is sometimes damaged due to disease or injury, or may simply deteriorate due to age, disease, injury or congenital defect. With others, the vertebrae may become compressed, misaligned or otherwise damaged. In these and other cases the vertebrae can become too closely spaced anteriorly which causes an undesired abnormal curvature of the spine with respect to lordosis or kyphosis.

Because of this, surgery may be utilized to place one or more spinal spacers or interbody devices between adjacent vertebrae in order to provide proper spacing of the vertebrae. In some cases the spinal interbody device may be an artificial or prosthetic spinal disc that is designed to replace the existing spinal disc. In some cases the spinal interbody device is a device that supports and/or realigns the adjacent vertebrae relative to one another and that promotes fusion between the vertebrae. Typically, bone fusion material is often placed about or in the spinal interbody device in order to promote growth of the bone between the adjacent vertebrae and thereby create fusion of the adjacent vertebrae.

The spinal interbody device is implanted between adjacent vertebrae where a spinal or spine disc should be (spinal disc cavity). Because such adjacent vertebrae are compressed, misaligned or otherwise damaged, the height of the opening (vertebrae opening) into which the interbody device must be implanted is narrow. This constraint restricts the height of an interbody device as it is being inserted into the spinal disc cavity. Since, however, the surgical goal is to expand the spinal disc cavity, the interbody device must either be or achieve a certain height after implantation into the spinal disc cavity. This height is greater than the vertebrae opening. Since these two dimensions are opposite, prior art spinal interbody devices have been developed that are designed to mechanically expand within the spinal disc cavity once implanted in order to provide distraction of adjacent vertebrae. Such spinal interbody devices are known as expandable spinal interbody devices.

These expandable interbody devices, however, involve moving parts. While these moving parts provide expandability of the interbody device once implanted, they also cause problems. In addition to moving parts, such expandable interbody devices are typically complicated structures causing them to be expensive to produce and/or prone to problems.

Accordingly, there presently exists a need for an improved spinal interbody device.

### SUMMARY OF THE INVENTION

The present invention is a spinal interbody distraction device (distractor) that is configured to be positionable through rotation (i.e. from 90° to 180°) of its entire body after implantation thereof into a spinal disc cavity of a patient's spine to provide distraction of adjacent vertebrae. Such implantation is preferably, but not necessarily, through minimally invasive surgery or surgical techniques.

The present spinal interbody distraction device has a lateral side to lateral side height of a first length and a superior end to inferior end height of a second length that is greater than the first length. This configuration allows the present spinal interbody distraction device to be inserted into a spinal disc cavity having an opening with a height that is smaller than an end height of the spinal disc cavity after implantation of the spinal interbody device thereby providing distraction of the adjacent vertebrae. The present spinal interbody distraction device is preferably, but not necessarily, for spinal fusion applications. As such, the spinal interbody distractor body may have one or more cavities, areas, openings and/or bores.

The present spinal interbody distraction device is made from a biocompatible material such as a thermoplastic (e.g. PEEK), a polymer, metal, combination thereof or otherwise, such as desired and/or is appropriate. The present spinal interbody distraction device may be used as and considered a spinal cage with the use of bone fusion material.

In one form according to the present principles, the body is defined by a generally H-shaped body having an arched anterior side. An upper fusion material cavity is defined by the H body that provides communication between the superior surface of the body (and thus the superior vertebrae of adjacent vertebrae) and the posterior end of the body. A lower fusion material cavity is defined by the H body that provides communication with the inferior surface of the body (and thus the inferior vertebrae of the adjacent vertebrae) and the posterior end of the body. In this manner, the cavities allow introduction of fusion material into and around the body from the posterior thereof.

The spinal interbody distraction device includes first and second gripping surfaces on the superior side of the body that are each configured to engage portions of the superior side vertebra of the adjacent vertebrae. Third and fourth gripping surfaces are provided on the inferior side of the body that are each configured to engage portions of the inferior side vertebra of the adjacent vertebrae.

In another form according to the present principles, the body is defined by a generally elongated rectangular body having an arched anterior side and is profiled in the lateral view to better fit the curvature of the vertebral bodies. A fusion material cavity is defined within the elongated rectangular body that is in communication with an opening in the posterior end of the body. The superior side of the body includes two openings each of which is in communication with the fusion material cavity. The inferior side of the body likewise includes two openings each of which is in communication with the fusion material cavity. The superior side openings and the inferior side openings provide communication between the superior surface of the body (and thus the superior vertebra of adjacent vertebrae) and the inferior surface of the body (and thus the inferior vertebra of the adjacent vertebrae, and the posterior end of the body.

The spinal interbody distraction device includes first and second gripping surfaces on the superior side of the body that are each configured to engage portions of the superior side vertebrae of the adjacent vertebrae. Third and fourth gripping surfaces are provided on the inferior side of the body that are each configured to engage portions of the inferior side vertebra of the adjacent vertebrae.

The present spinal interbody distractor may be made in various sizes both as to height and width ratios, and to overall dimensions. As such, one or more of the present spinal interbody distractors may be used between the same adjacent vertebrae.

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a posterior perspective view of an exemplary embodiment of a spinal interbody distraction device fashioned in accordance with the present principles;

FIG. 2 is an anterior perspective view of the spinal interbody distraction device of FIG. 1;

FIG. 3 is an inferior view of the rotatable spinal interbody distraction device of FIG. 1;

FIG. 4 is a lateral side view of the rotatable spinal interbody distraction device of FIG. 1;

FIG. 5 is a posterior perspective view of another exemplary embodiment of a spinal interbody distraction device fashioned in accordance with the present principles;

FIG. 6 is a posterior end view of the spinal interbody distraction device of FIG. 5;

FIG. 7 is a superior end view of the spinal interbody distraction device of FIG. 5; and

FIG. 8 is a lateral side view of the spinal interbody distraction device of FIG. 5.

Like reference numerals indicate the same or similar parts throughout the several figures.

A detail of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF EMBODIMENTS OF THE INVENTION

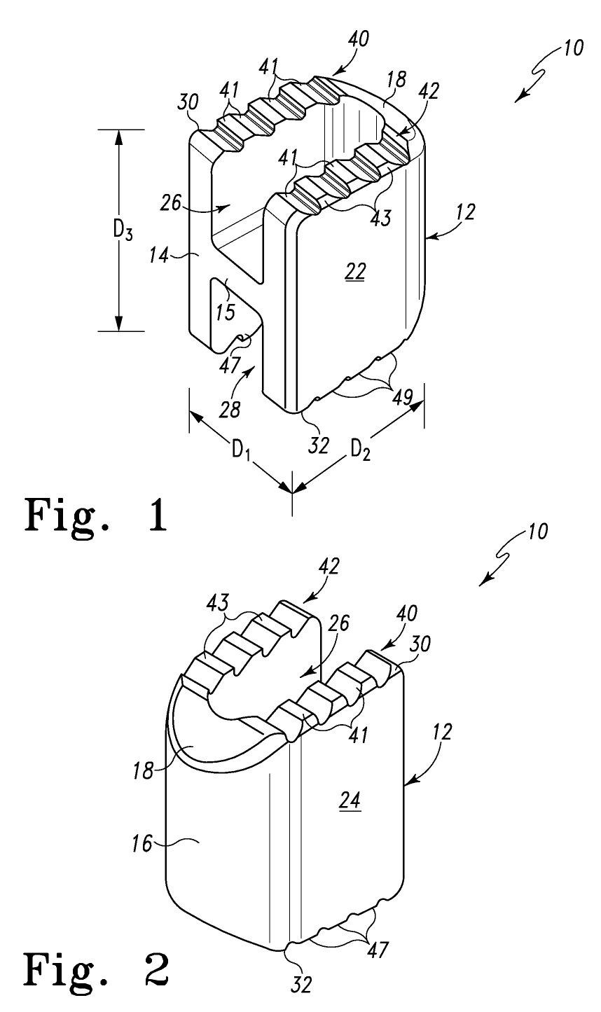

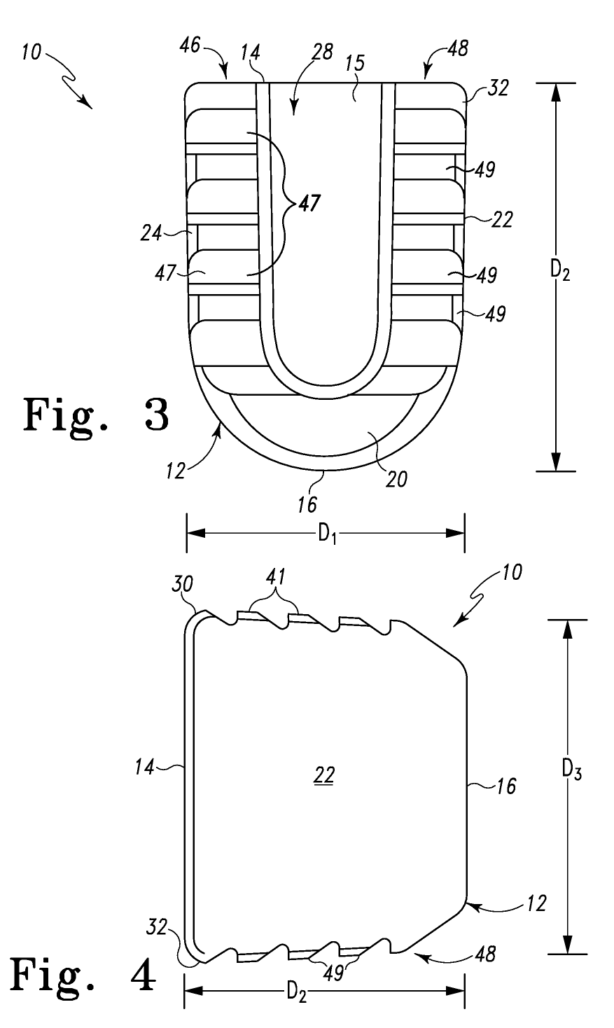

Referring to FIGS. 1-4 there is depicted an exemplary embodiment of a spinal interbody distraction device or distractor, generally designated 10, fashioned in accordance with the present principles. The spinal interbody distraction device 10 is characterized by a body 12 in the general shape of an H but having an arched or arced anterior end, side or surface 16. The body 12 has a posterior end, side or surface 14 in the shape of an H and thus defines a cross member 15. In one form as shown, the cross member 15 provides a division between upper and lower cavities 26 and 28. In another form, the cross member 15 forms a partial division between the upper and lower cavities 26 and 28. This form would allow and/or promote through growth. The body 12 may be fabricated in various sizes to accommodate various applications.

The body 12 also defines a first lateral side surface 22 that extends from the posterior end 14 to the anterior end 16 on one side of the body 12, and a second lateral side surface 24 that extends from the posterior end 14 to the anterior end 16 an another side of the body 12. The body 12 further defines a superior end, side or surface 30 and an inferior end, side or surface 32. The superior end 30 has spaced apart vertebral body contact or gripping portions or strips 40 and 42 that extend from the posterior end 14 to the anterior end 16 and transition to the anterior end via an arcuate taper 18. The inferior end 32 has spaced apart vertebral body contact or gripping portions or strips 46 and 48 that extend from the posterior end 14 to the anterior end 16 and transition to the anterior end via an arcuate taper 20.

The gripping portion 40 of the superior side 30 is characterized by a plurality of teeth or teeth structures 41 that define plateaus and valleys for contact with a vertebral body. The plurality of teeth 41 are configured to allow insertion of the body 12 via the anterior end thereof, but prevent backing out. In this regard, the teeth 41 are oriented accordingly (see e.g. FIG. 4). Likewise, the gripping portion 42 of the superior side 30 is characterized by a plurality of teeth or teeth structures 43 that define plateaus and valleys for contact with a vertebral body. The plurality of teeth 43 are configured to allow insertion of the body 12 via the anterior end thereof, but prevent backing out. In this regard, the teeth 43 are oriented accordingly.

The gripping portion 46 of the inferior side 32 is characterized by a plurality of teeth or teeth structures 47 that define plateaus and valleys for contact with a vertebral body. The plurality of teeth 47 are configured to allow insertion of the body 12 via the anterior end thereof, but prevent backing out. In this regard, the teeth 47 are oriented accordingly. Likewise, the gripping portion 48 of the inferior side 32 is characterized by a plurality of teeth or teeth structures 49 that define plateaus and valleys for contact with a vertebral body. The plurality of teeth 49 are configured to allow insertion of the body 12 via the anterior end thereof, but prevent backing out. In this regard, the teeth 49 are oriented accordingly (see e.g. FIG. 4).

Referring to FIGS. 1, 3 and 4, the body 12 defines various dimensions D₁, D₂ and D₃. Dimension D₁ is a lateral side to lateral side length or height of the body 12. Dimension D₂ is a posterior end to anterior end length of the body 12. Dimension D₃ is a superior end to inferior end length or height of the body 12. The length or height of D₁ is less than the length or height of D₃. This allows the body 12 to be implanted into a spinal disc cavity having a height or length of D₁ and then achieve a spinal disc cavity height or length of D₃ that is greater than D₁.

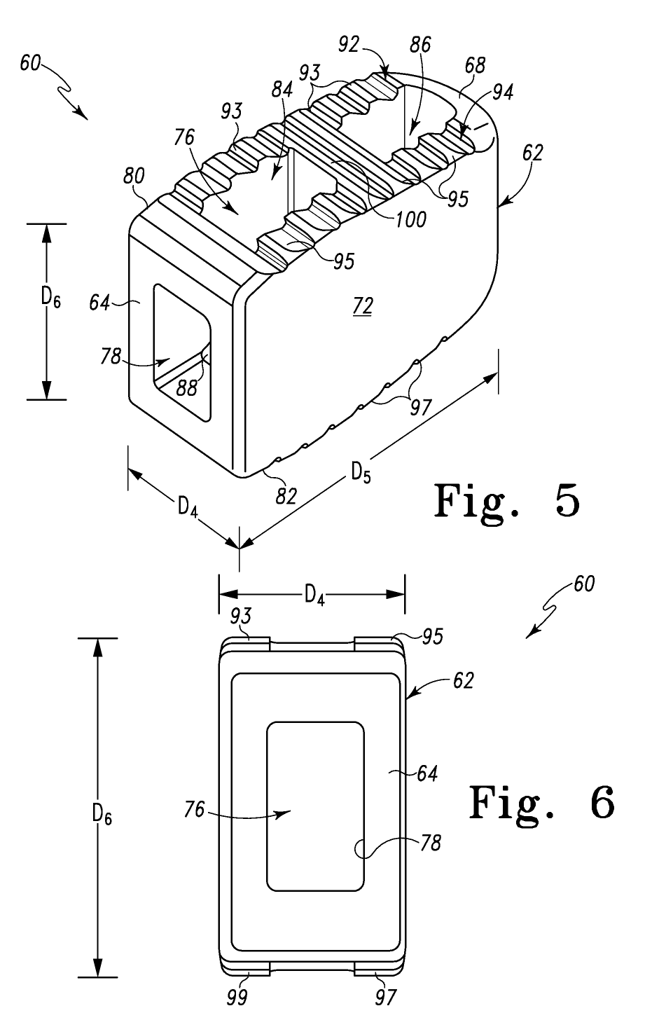

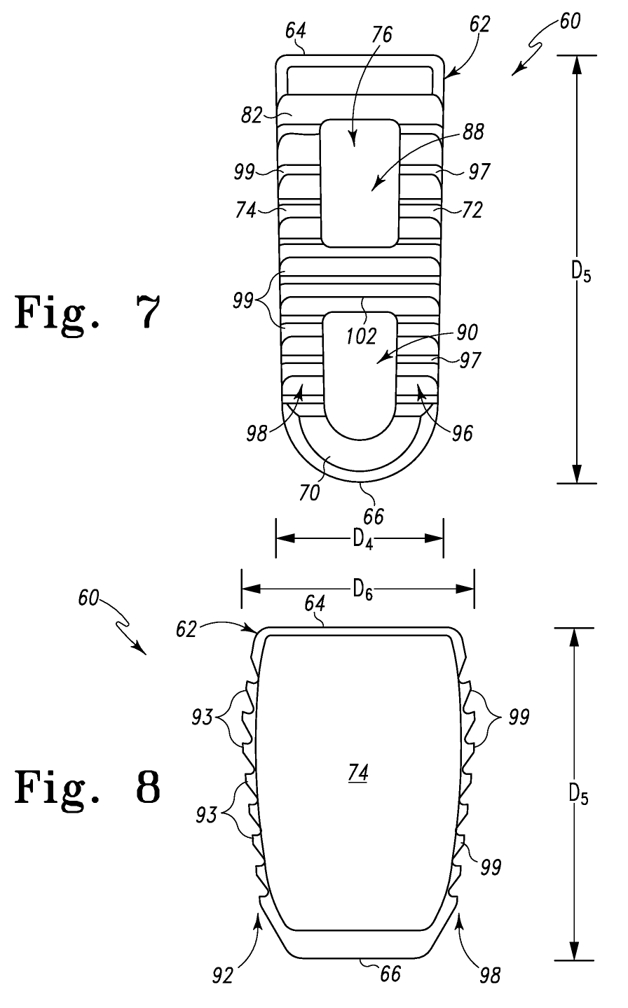

Referring to FIGS. 5-8 there is depicted another exemplary embodiment of a spinal interbody distraction device, generally designated 60, fashioned in accordance with the present principles. The spinal interbody distraction device 60 is characterized by a body 62 in the general shape of an elongated rectangle but having an arched or arced anterior end, side or surface 66. The body 62 has a posterior end, side or surface 64 having an opening 78 therein that is in communication with an interior cavity 76. The body 62 may be fabricated in various sizes to accommodate various applications. The body 62 also defines a first lateral side surface 72 that extends from the posterior end 64 to the anterior end 66 on one side of the body 62, and a second lateral side surface 74 that extends from the posterior end 64 to the anterior end 66 an another side of the body 62.

The body 62 further defines a superior end, side or surface 80 and an inferior end, side or surface 82. A first superior side opening 84 is provided which is in communication with the interior cavity 76. A second superior side opening 86 is provided which is also in communication with the interior cavity 76. The superior end 80 also has spaced apart vertebral body contact or gripping portions or strips 92 and 94 that extend from the posterior end 64 to the anterior end 66 and transition to the anterior end via an arcuate taper 68. The first and second openings 84 and 86 are separated by a bridge portion 100. A first inferior side opening 88 is provided which is in communication with the interior cavity 76. A second inferior side opening 90 is provided which is also in communication with the interior cavity 76. The inferior end 82 has spaced apart vertebral body contact or gripping portions or strips 96 and 98 that extend from the posterior end 64 to the anterior end 66 and transition to the anterior end via an arcuate taper 70. The first and second openings 88 and 90 are separated by a bridge portion 102.

The gripping portion 92 of the superior side 80 is characterized by a plurality of teeth or teeth structures 93 that define plateaus and valleys for contact with a vertebral body. The plurality of teeth 93 are configured to allow insertion of the body 62 via the anterior end thereof, but prevent backing out. In this regard, the teeth 93 are oriented accordingly (see e.g. FIG. 8). Likewise, the gripping portion 94 of the superior side 80 is characterized by a plurality of teeth or teeth structures 95 that define plateaus and valleys for contact with a vertebral body. The plurality of teeth 95 are configured to allow insertion of the body 62 via the anterior end thereof, but prevent backing out. In this regard, the teeth 95 are oriented accordingly.

The gripping portion 96 of the inferior side 82 is characterized by a plurality of teeth or teeth structures 97 that define plateaus and valleys for contact with a vertebral body. The plurality of teeth 97 are configured to allow insertion of the body 62 via the anterior end thereof, but prevent backing out. In this regard, the teeth 97 are oriented accordingly. Likewise, the gripping portion 98 of the inferior side 82 is characterized by a plurality of teeth or teeth structures 99 that define plateaus and valleys for contact with a vertebral body. The plurality of teeth 99 are configured to allow insertion of the body 62 via the anterior end thereof, but prevent backing out. In this regard, the teeth 99 are oriented accordingly (see e.g. FIG. 8).

The body 62 defines various dimensions D₄, D₅ and D₆. Dimension D₄ is a lateral side to lateral side length or height of the body 62. Dimension D₅ is a posterior end to anterior end length of the body 62. Dimension D₆ is a superior end to inferior end length or height of the body 62. The length or height of D₄ is less than the length or height of D₆. This allows the body 62 to be implanted into a spinal disc cavity having a height or length of D₄ and then achieve a spinal disc cavity height or length of D₆ that is greater than D₄. The body 62 is profiled in the lateral view to better fit the curvature of the vertebral bodies.

The configuration of the bodies 12 and 62 as described above allows the body to be inserted into the spinal disc cavity or space between adjacent vertebras with its lateral sides facing the adjacent vertebrae via minimally invasive surgery (i.e. a minimally invasive surgical technique). After insertion, the body is rotated from 90° to 180° such that the superior end and the inferior end abut and/or face the adjacent vertebrae. After rotation, the height of the spinal disc cavity is greater than before thereby providing distraction of the adjacent vertebrae. Bone fusion material may then be provided to the device as necessary.

The bodies 12 and 62 are preferably, but not necessarily, made from PEEK (polyetheretherketone) but may be made from other biocompatible thermoplastics. It should be appreciated that the body may take other shapes as desired and/or as is appropriate and which are fashioned in accordance with the present principles. As well, the body may be formed from other biocompatible materials such as biocompatible thermoplastics, polymers, metals or materials as desired and/or as is appropriate.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A spinal interbody distractor comprising:
a body formed of a biocompatible material and defining first and second lateral sides, a superior side, an inferior side, a first length defined between the first and second lateral sides, and a second length defined between the superior side and the inferior side, the second length being greater than the first length, whereby rotation of the body by 90° to 180° after implantation within a spinal disc cavity of adjacent vertebrae provides distraction of the adjacent vertebrae;
a first plurality of serrations disposed on the superior side of the body; and
a second plurality of serrations disposed on the inferior side of the body.

2. The spinal interbody distractor of claim 1, wherein the body further defines a posterior side that is curved from the first lateral side to the second lateral side.

3. The spinal interbody distractor of claim 2, wherein the body further defines an anterior side that is planar.

4. The spinal interbody distractor of claim 3, wherein the body is configured as in an H shape.

5. The spinal interbody distractor of claim 3, wherein the body is configured as an elongated rectangle.

6. The spinal interbody distractor of claim 1, wherein the body further defines a plurality of cavities formed therein.

7. The spinal interbody distractor of claim 1, wherein the body is formed of PEEK.

8. The spinal interbody distractor of claim 1, wherein:
the first plurality of serrations are angled from the posterior side to the anterior side of the body; and
the second plurality of serrations are angled from the posterior side to the anterior side of the body.

9. The spinal interbody distractor of claim 8, wherein:
the first plurality of serrations extend along the superior side from the posterior side to the anterior side of the body; and
the second plurality of serrations extend along the inferior side from the posterior side to the anterior side of the body.

10. A spinal interbody distractor comprising:
a unitary body formed of a biocompatible material and defining first and second lateral sides, a superior side, an inferior side, a first length defined between the first and second lateral sides, and a second length defined between the superior side and the inferior side, the second length being greater than the first length, whereby rotation of the body by 90° to 180° after implantation within a spinal disc cavity of adjacent vertebrae provides distraction of the adjacent vertebrae;
a first plurality of serrations disposed on the superior side of the body and extending from the posterior side to the anterior side; and
a second plurality of serrations disposed on the inferior side of the body and extending from the posterior side to the anterior side.

11. The spinal interbody distractor of claim 10, wherein the body further defines a posterior side that is curved from the first lateral side to the second lateral side.

12. The spinal interbody distractor of claim 11, wherein the body further defines an anterior side that is planar.

13. The spinal interbody distractor of claim 12, wherein the body is configured as in an H shape.

14. The spinal interbody distractor of claim 12, wherein the body is configured as an elongated rectangle.

15. The spinal interbody distractor of claim 10, wherein the body further defines a plurality of cavities formed therein.

16. The spinal interbody distractor of claim 10, wherein the body is formed of PEEK.

17. The spinal interbody distractor of claim 10, wherein:
the first plurality of serrations are angled from the posterior side to the anterior side of the body; and
the second plurality of serrations are angled from the posterior side to the anterior side of the body.

18. A method of distracting adjacent vertebrae comprising the steps of:
introducing a spinal interbody distractor comprising, i) a body formed of a biocompatible material and defining first and second lateral sides, a superior side, an inferior side, a first length defined between the first and second lateral sides, and a second length defined between the superior side and the inferior side, the second length being greater than the first length, ii) a first plurality of serrations disposed on the superior side of the body, and iii) a second plurality of serrations disposed on the inferior side of the body, into a spinal disc cavity of adjacent vertebrae; and
rotating the spinal interbody distractor from 90° to 180° after implantation within the spinal disc cavity of adjacent vertebrae to provide distraction of the adjacent vertebrae.
